(19) United States
(12) Patent Application Publication
(10) Pub. No.: US 2026/0366965 A1
(43) Pub. Date: Nov. 19, 2026

(54) ON-LINE ANALYSIS SYSTEM AND METHOD FOR SPECIALTY GASSES

(71) Applicant: Taiwan Semiconductor Manufacturing Co., Ltd., Hsinchu (TW)

(72) Inventors: Chiang Jeh CHEN, Hsinchu (TW); Chieh-Jan HUANG, Hsinchu (TW); Lien-Fang WU, Hsinchu (TW)

(73) Assignee: Taiwan Semiconductor Manufacturing Co., Ltd., Hsinchu (TW)

(21) Appl. No.: 19/752,184

(22) Filed: Jul. 23, 2026

Related U.S. Application Data
(62) Division of application No. 17/752,730, filed on May 24, 2022.

(51) Int. Cl. G01N 1/22; G01N 1/10

(52) U.S. Cl. CPC G01N 1/2226; G01N 1/10; G01N 2001/1018; G01N 2001/2285

(57) ABSTRACT

The present disclosure includes methods and systems for monitoring real time quality of specialty fluids within one or more specialty fluid containers stored within one or more gas cabinets. The methods and systems monitoring the real time quality of the specialty fluids may utilize an analyzer and sampler to perform one or more tests on the specialty fluids to determine the quality of the specialty fluids in real time. The data collected by the analyzer and sampler may be stored on and processed with the on-line data system to determine if the quality of the specialty fluids is sufficient to be introduced to one or more workpiece processing tools for processing one or more workpieces. The data collected may be compared to a manufacturer technical specification with respect to the specialty fluid to determine if the manufacturer is providing specialty fluids of sufficient quality.

### PRIORITY CLAIM

This application is a divisional of U.S. Patent Application No. 17/752,730, filed on May 24, 2022, which is incorporated by reference herein in its entirety.

### BACKGROUND

Generally, in the manufacture of semiconductor devices or packages within a semiconductor manufacturing plant (FAB), various specialty gasses (e.g., liquids or gasses) are stored within storage containers within the FAB. The various specialty gasses may be fluidically communicated to various workpiece processing tools such that the workpiece processing tools may utilize the specialty gas to perform processing steps (e.g., etching steps, patterning steps, etc.) on workpieces (e.g., semiconductor wafers, silicon wafers, etc.). A plurality of fluid pathways (e.g., pipes) are present within the FAB such that the specialty gas may be transported throughout the FAB. The fluid pathways are configured to transport the specialty gas within the FAB from specialty fluid containers (e.g., liquid containers, gas containers, etc.) to the workpiece processing tools, and the workpiece processing tools utilize the gas in some fashion to refine or process respective workpieces at the workpiece processing tools to manufacture semiconductor devices or packages.

The storage fluid containers may be gas cylinders that contain specialty fluids in a gaseous state. Once all or the majority of the gaseous state fluid within a respective one of the storage fluid containers is utilized such that the respective one of the storage fluid containers is empty, the respective one of the storage fluid container may be replaced with a new storage fluid container that is full with specialty fluid in a gaseous state.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A illustrates an example of a system to test specialty fluids within specialty fluid containers.

FIG. 1B illustrates an example of a flowchart of a method for testing the specialty fluids in the specialty fluid containers as shown in FIG. 1A.

FIG. 2 illustrates an example of a system to test specialty fluids, in accordance with some embodiments.

FIG. 3 illustrates an example of a gas cabinet of the system to test specialty fluids within specialty fluid containers as shown in FIG. 2, in accordance with some embodiments.

FIG. 4 illustrates an example of a gas cabinet of the system to test specialty fluids within specialty fluid containers as shown in FIG. 2, in accordance with some embodiments.

FIG. 5 illustrates an example of an analyzer and sampler system or tool in fluidic communication with the gas cabinet as shown in FIG. 3, in accordance with some embodiments.

FIG. 6 illustrates an example of a flowchart of a method of utilizing the system to test specialty fluids within specialty fluid containers as shown in FIG. 2, in accordance with some embodiments.

FIG. 7 illustrates an example of a flowchart of a control block diagram to monitor and test specialty fluids within specialty fluid containers as shown in FIG. 2 in real time, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed

Further, spatially relative terms, such as “beneath,” “below,” “lower,” “above,” “upper” and the like, may be used herein for ease of description to describe one element or feature’s relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Generally, manufacturers that sell and manufacture specialty fluids (e.g., liquid state, gaseous state, semi-liquid state, semi-gaseous state, etc.) store those specialty fluids within specialty fluid containers (e.g., liquid cylinders, liquid canisters, gas cylinders, gas canisters, etc.) for shipment and utilization within a semiconductor manufacturing plant (FAB). The manufacturers provide manufacturer technical specifications for the specialty gas stored within the specialty fluid containers. For example, the manufacturer technical specification with respect to the specialty fluid may include characteristics of the specialty fluid within the specialty fluid containers such as purity, contaminant levels, amount of specialty fluid, type of specialty fluid, composition of specialty fluid, or some other similar or like types of characteristics that may be useful information to a manufacturer of semiconductor devices or packages that intends to utilize the specialty fluid provided by the manufacturer of the specialty fluid. However, as manufacturing semiconductor devices or packages is susceptible to contaminants, at least some of the specialty fluids in some of the specialty fluid storage containers may be tested utilizing specialty fluid analysis tools, devices, or systems to determine the accuracy of the manufacturer’s technical specification to reduce the likelihood of utilizing sub-quality or overly contaminated specialty fluids while manufacturing semiconductor device or packages. In other words, these tests may be carried out to avoid exposing workpieces to specialty fluids of insufficient quality (e.g., too high of a contaminant level).

For example, a manufacturer may assert that a specialty fluid in a specialty fluid container is within prescribed tolerances, for example, below a specified contaminant level such that the specialty fluid is of a high quality and is sufficient quality for utilization to manufacture semiconductor devices or packages without generating defective ones of the semiconductor devices or packages due to contaminants present within the specialty fluid. However, if the contaminants present within the specialty fluid is greater than as asserted by the manufacturer in the manufacturer’s technical specification, several defective or below tolerance semiconductor devices or packages may be manufactured due to the specialty fluid of insufficient quality (e.g., contains too many contaminants) being utilized during a step in a manufacturing process to manufacture the semiconductor devices or packages within a FAB. Manufacturing defective, deficient, or out of tolerance ones of the semiconductor devices or packages generally increases costs due to increased levels of scrap as the defective, deficient, or out of tolerance ones of the semiconductor devices or packages are of insufficient quality to be sold to customers.

In view of the above, while the manufacturer technical specification may assert that the specialty fluid is of sufficient quality and within appropriate tolerances as needed to manufacture the semiconductor devices or packages within the FAB, in actuality, the specialty fluid within the specialty fluid containers may not be of sufficient quality or within appropriate tolerances. Accordingly, as the manufacturer’s technical specification for the specialty fluid may be inaccurate at least upon receipt by the manufacturer of the semiconductor devices or packages of the specialty fluid container containing the specialty fluid, if the manufacturer of the semiconductor devices or packages utilizes the specialty fluid of insufficient quality during the manufacturing process of the semiconductor devices or packages, a number of defective semiconductor devices or packages that are manufactured outside of tolerances increases, and, therefore, results in greater scrap costs that are absorbed by the manufacturer of the semiconductor devices or packages.

In view of the above, at least some of the present disclosure is directed to systems and methods for testing and determining quality levels (e.g., contaminant levels) of specialty fluids stored within specialty fluid containers that are to be utilized in manufacturing the semiconductor devices or packages by the manufacturer of the semiconductor devices or packages. This testing to confirm that the specialty fluid is of sufficient quality reduces the likelihood of manufacturing deficient or defective ones of the semiconductor devices or packages, and, therefore, the scrap costs absorbed by the manufacturer of the semiconductor devices or packages is reduced. Data collected from running these tests on the specialty fluids within the specialty fluid containers may be utilized to generate technical specifications for the specialty fluid in the specialty fluid containers to monitor the quality of the specialty fluids received by the manufacturer of the semiconductor devices and packages for quality control purposes and to monitor the quality of the specialty fluids provided to the manufacturer of the semiconductor devices or packages.

The present disclosure is further directed to systems and methods for testing the specialty fluids within the specialty fluid containers in real time such that the real time quality of the specialty fluids is monitored to avoid introducing the specialty fluids to workpiece processing tools to perform processing steps on a workpiece in an instance in which the specialty fluid becomes of insufficient quality at a later time. Monitoring the real time quality of the specialty fluid before or when being utilized by the workpiece processing tool, the likelihood of exposing the workpiece to insufficient quality specialty fluid is reduced, which again reduces scrap costs, material costs, and increases the likelihood of manufacturing semiconductor devices or packages within tolerances that may be sold and shipped to customers.

The updated technical specifications may be utilized in systems and methods that monitor the specialty fluids in the specialty fluid containers in real time, in accordance with some embodiments of the present disclosure. For example, these real time monitoring systems and methods that monitor the quality of the specialty fluids in real time when in use may utilize the updated technical specifications to provide more accurate determinations than when the manufacturer technical specifications are utilized instead. For example, one such determination may be that a respective specialty fluid container containing a respective specialty fluid has dropped below sufficient quality and needs to be replaced. These methods and systems structured and configured to monitor real time quality (e.g., contaminant levels, composition, etc.) of the specialty fluids in the specialty fluid containers are utilized to reduce the likelihood of exposing workpieces to specialty fluids of insufficient quality that may result in the manufacturing of deficient or defective semiconductor devices or packages. This exposure of workpieces to specialty fluid of insufficient quality again may at least increase scrap costs and material costs that are generally absorbed by the manufacturer of the semiconductor devices or packages.

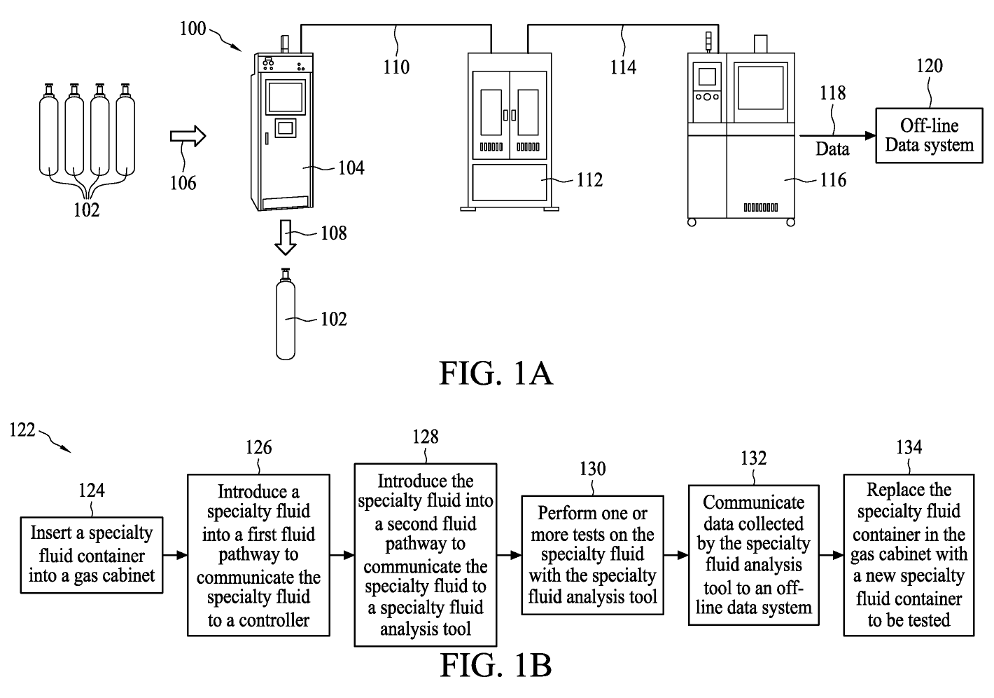

FIG. 1A illustrates an example of a system 100 to test specialty fluids within specialty fluid containers 102. As shown in FIG. 1A, the system 100 is a system that is “off-line” from workpiece processing tools within a semiconductor manufacturing plant (FAB). The system 100 is “off-line” in that when testing the specialty fluids within the specialty fluid containers 102 with the testing system 100, the specialty fluid within the specialty fluid containers 102 cannot be utilized by the workpiece processing tools within the FAB when being tested by the system 100.

As shown in FIG. 1A, the off-line testing system 100 includes a gas cabinet 104, which is not in fluid communication with the other workpiece processing tools within the FAB such that the specialty fluids contained within the specialty fluid containers 102 being tested utilizing the system 100 are not capable of being communicated to the workpiece processing tools within the FAB as the system 100 is off-line from the workpiece processing tools within the FAB. The gas cabinet 104 receives one of the specialty fluid containers 102 for testing. The insertion of one of the specialty fluid containers 102 into the gas cabinet 104 is represented by an arrow 106, respectively. After the specialty fluid is tested within the one of the specialty fluid containers 102 when present in the gas cabinet 104, the one of the specialty fluid containers 102 is removed from the gas cabinet 104 as represented by the arrow 108, respectively. Details of testing the specialty fluids within the specialty fluid containers 102 will be discussed further with respect to FIG. 1B as follows later herein within the present disclosure.

A first fluid pathway 110 extends from the gas cabinet 104 to a controller and/or selector 112, which may be referred to as a controller/selector, such that the one of the gas cabinet 104 is in fluid communication with the controller 112 through the first fluid pathway 110. A second fluid pathway 114 extends from the controller 112 to a specialty fluid analysis tool 116. The specialty fluid analysis tool 116 is in fluid communication with the controller 112 through the second fluid pathway 114. The specialty fluid within the one of the specialty fluid containers 102 present within the gas cabinet 104 being tested allows for the specialty fluid to be fluidically communicated through the first fluid pathway 110 to the controller 112 from the gas cabinet 104 and through the second fluid pathway 114 to the specialty fluid analysis tool 116 from the controller 112. The specialty fluid analysis tool 116 is configured to perform one or more types of tests on the specialty fluid received by the specialty fluid analysis tool 116 through the second fluid pathway 114. Data 118 collected by the specialty fluid analysis tool 116 is transmitted to an off-line data system 120 that is off-line in a similar or like manner as the system 100 in that the off-line data system 120 is not in electrical communication with the workpiece processing tools or a database system that is in electrical communication with the workpiece processing tools. In other words, the system 100 as shown in FIG. 1A does not monitor real time data of the specialty fluids within the specialty fluid containers 102 when in use with the workpiece processing tools within the FAB.

FIG. 1B is directed to a flowchart 122 of a method utilizing the system 100 to perform one or more types of tests on the specialty fluids within the specialty fluid containers 102, respectively. As shown in FIG. 1B, the flowchart 122 includes a first step 124, a second step 126, a third step 128, a fourth step 130, a fifth step 132, and a sixth step 134.

In the first step 124, a respective one of the specialty fluid containers 102 is inserted into the gas cabinet 104. For example, an employee of the FAB may transport the respective one of the specialty fluid containers 102 to the gas cabinet 104 and insert the respective one of the specialty fluid containers 102 into the gas cabinet 104. As discussed earlier, the insertion of the respective one of the specialty fluid containers 102 is represented by the arrow 106

After the first step 124 in which the respective one of the specialty fluid container 102 is inserted and mounted within the gas cabinet 104, in the second step 126 the employee may actuate a valve at an upper end of the respective one of the specialty fluid containers 102 such that the specialty fluid may readily exit the respective one of the specialty fluid containers 102 and enter into the first fluid pathway 110. The specialty fluid that exits the respective one of the specialty fluid containers 102 and enters into the first fluid pathway 110 passes through the first fluid pathway to the controller 112 to introduce the specialty fluid into the controller 112. The specialty fluid may be collected by the controller in a controlled manner. For example, the controller may collect a first amount of the specialty fluid, which may be referred to as a sample of the specialty fluid.

After the second step 126 in which the specialty fluid from the respective one of the specialty fluid containers 102 is provided to the controller 112 and the controller collects the sample, in the third step 128 the controller 112 allows for the sample to enter the second fluid pathway 114 to be communicated to the specialty fluid analysis tool 116. For example, the controller 112 may include a sample valve (not shown) that is in fluid communication with the second fluid pathway 114, and, after the sample has been collected by the controller 112, the controller 112 may open the sample valve such that the sample passes into and through the second fluid pathway 114 to the specialty fluid analysis tool 116.

After the third step 128 in which the sample of the specialty fluid from the respective one of the specialty fluid containers 102 is communicated through the second fluid pathway 114 to the specialty fluid analysis tool 116, in the fourth step 130 the specialty fluid analysis tool 116 performs one or more tests on the sample utilizing one or more sensors (not shown) or testing tools (not shown).

After the fourth step 130 in which the one or more tests are performed on the sample by the specialty fluid analysis tool 116, in the fifth step 132 data collected by the specialty fluid analysis tool 116 that performed the one or more tests of the sample is electrically communicated to the off-line data system 120, which may be a memory. As discussed earlier, the off-line data system 120 is not in electrical communication with the one or more workpiece processing tools or other electronic devices or components within the FAB other than the specialty fluid analysis tool 116.

After the fifth step 132 in which the data is collected and stored in the off-line data system 120, in a sixth step 134 the respective one of the specialty fluid containers 102 that has been tested is replaced with a new, successive one of the specialty fluid containers 102. The method as shown in the flowchart 122 may then be carried out successively on the new, successive one of the specialty fluid containers 102 to test the specialty fluid within the new, successive one of the specialty fluid containers. In other words, the method of the flowchart 122 may be carried out successively multiple times to test specialty fluids within the specialty fluid containers 102, respectively.

After the respective one of the specialty fluid containers 102 that was previously tested and has been replaced by the new, successive one of the specialty fluid containers 102 is removed from the cabinet 104 and replaced, the respective one of the specialty fluid containers 102 is discarded instead of being utilized within the FAB to manufacture semiconductor devices or packages within the FAB. The specialty fluid container 102 is discarded as contaminants may have entered the respective one of the specialty fluid container 102 when the specialty fluid container 102 is removed from the gas cabinet 104 by the employee of the FAB. For example, if the valve at the upper end of the respective one of the specialty fluid containers 102 is not fully closed, contaminants may enter through the valve at the upper end of the respective one of the specialty fluid containers 102 such that utilizing the specialty fluid container may result in manufacturing an increased number of deficient or defective ones of semiconductor devices or packages utilizing the FAB. Discarding the respective one of the specialty fluid containers 102 results in the rest of the specialty fluid present within the respective one of the specialty fluid container 102 becoming waste. The result of discarding the specialty fluid containers 102 increases testing costs and waste costs, which limits a number of the specialty fluid containers 102 that are actually tested utilizing the system 100.

In view of the above discussion with respect to FIGS. 1A and 1B, as well as the deficiency in the discarding of the specialty fluid containers 102 containing fluid after testing utilizing the system 100, the present disclosure is directed to providing on-line system and on-line methods of testing to avoid discarding of the specialty fluid containers 102 that still contain specialty fluid, respectively, to reduce waste costs as well as to monitor real time characteristics of a specialty fluid when in actual use by the FAB to manufacture semiconductor devices and packages. In other words, the present disclosure is directed to providing embodiments of on-line testing systems that avoid the deficiencies of the system 100 as shown in FIGS. 1A and 1B as discussed above such that a yield of a number of semiconductor devices or packages that are manufactured within tolerances and are able to be sold and shipped to customers increases.

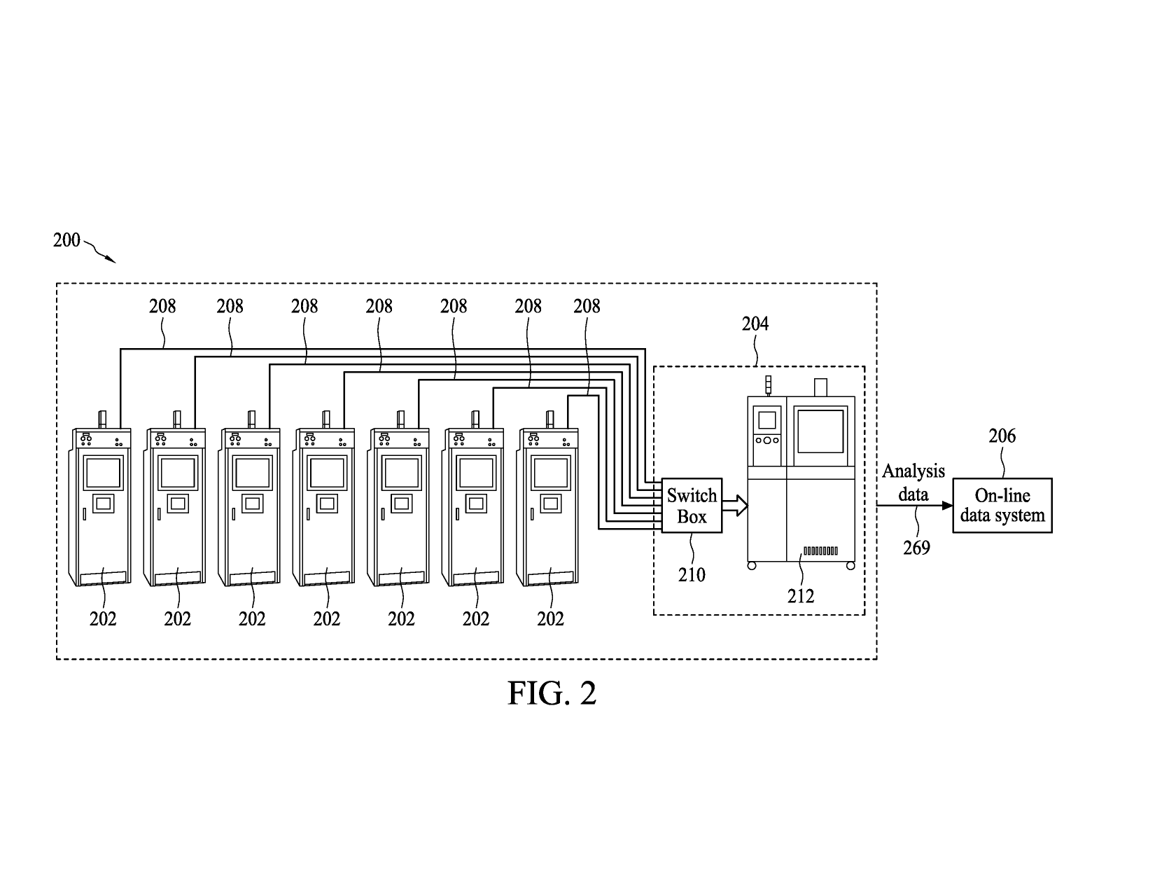

FIG. 2 illustrates an example of a system 200 to test specialty fluids, which may be stored in specialty fluid containers, in accordance with some embodiments. The system includes one or more gas cabinets 202, a specialty fluid analyzer and sampler 204, and an on-line data system 206. The fluid analyzer and sampler 204, which may be referred to as an on-line analysis system, may be a plurality of analyzer tools or sampler tools such that the fluid analyzer and sampler 204 may be a fluid analyzer and sampler system. For the sake of simplicity and brevity of the present disclosure, the specialty fluid analyzer and sampler 204 may be referred to as an analyzer 204 herein.

Each one of the one or more gas cabinets 202 are in fluid communication with the analyzer 204 through one or more test fluid pathways 208 that extend from each one of the one or more gas cabinets 202 to the analyzer 204. As shown in the embodiment in FIG. 2, there is a one-to-one relationship between a number of the one or more gas cabinets 202 and a number of the one or more test fluid pathways 208. However, in some embodiments, more than one of the one or more test fluid pathways 208 may extend from at least one of the one or more gas cabinets 202. In other words, the number of the one or more gas cabinets 202 may be less than the number of the one or more test fluid pathways 208. For example, two of the one or more test fluid pathways 208 may be in fluid communication with a left-most gas cabinet 202 as shown in FIG. 2 and the two of the one or more test fluid pathways 208 may extend to the analyzer 204 such that the left-most gas cabinet 202 is in fluid communication with the analyzer through the two of the one or more test fluid pathways 208, respectively.

The one or more test fluid pathways 208 have first ends at respective ones of the one or more gas cabinets 202 and have second ends, which are opposite to the first ends, at a switch box 210 of the analyzer 204. The switch box 210 is in fluid communication with the second ends of the one or more test fluid pathways 208, and the switch box 210 is in fluid communication with a specialty fluid analyzer, sampler, or testing tool 212. For the sake of simplicity and brevity of the present disclosure, the analyzer, sampler, or testing tool 212 may be referred to as a testing tool 212.

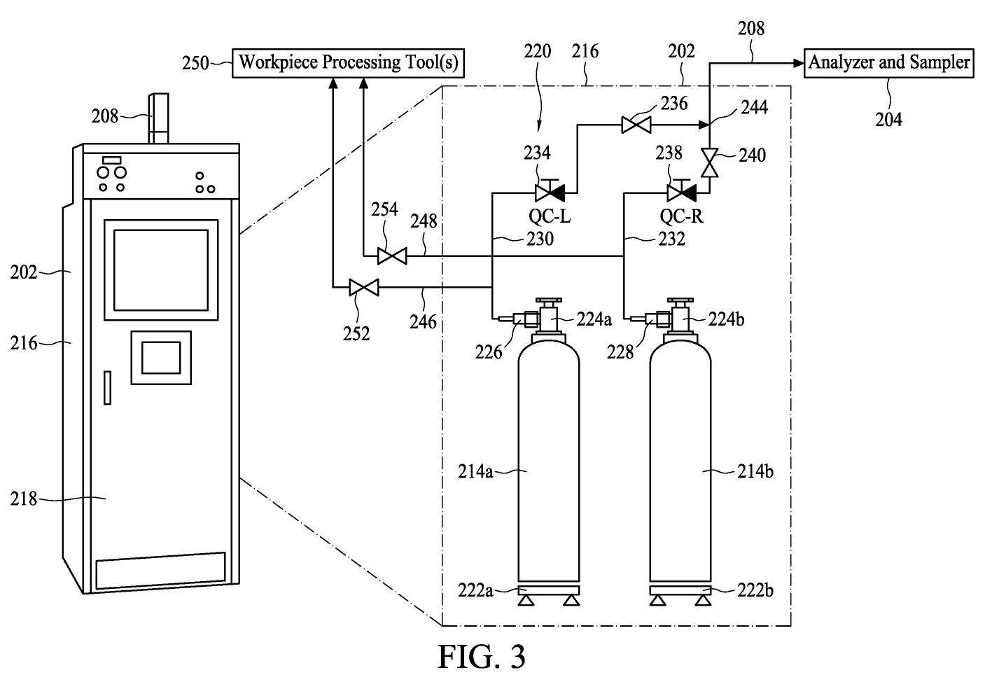

While not shown in FIG. 2, each one of the gas cabinets 202 may contain one or more fluid containers 214a, 214b (see a first fluid container 214a and a second fluid container 214b in FIG. 3 of the present disclosure) that each contain a specialty fluid that may be tested utilizing the testing tool 212 of the analyzer 204. For example, a valve (not shown in FIG. 2) may be opened such that the specialty fluid in at least one of the one or more fluid containers 214a, 214b is transported along at least one of the one or more test fluid pathways 208 to the switch box 210. The specialty fluid transported to the switch box 210 may then pass through the switch box 210 into the testing tool 212 such that the specialty fluid is received by the testing tool 212. Once the specialty fluid is received by the testing tool 212, the testing tool 212 may perform one or more types of tests on the specialty fluid to determine and ascertain selected characteristics of the specialty fluid. For example, a level of contaminants within the specialty fluid, a composition of the specialty fluid, or some other similar or like characteristics of the specialty fluid.

The one or more types of tests performed by the testing tool 212 on the specialty fluid received from the switch box 210 may include cavity ring-down spectroscopy (CRDS) tests, gas chromatography tests (e.g., pulsed helium ionization detection (PDHID) test, mass spectrometry (MS) test, etc.), IC tests, ToFMS tests, or some other similar or like types of tests or combinations of types of tests that may be performed on the specialty fluid by the testing tool 212. While not shown in detail, the testing tool 212 includes one or more types of sensors and testing components that are utilized to perform the one or more types of tests on the specialty fluid to ascertain or determine characteristics (e.g., contamination level, compositional information, etc.) of the specialty fluid as discussed above.

FIG. 3 illustrates an example of one of the one or more gas cabinets 202 of the system 200 as shown in FIG. 2, in accordance with some embodiments. While the following discussion will focus on the gas cabinet 202 as shown in FIG. 3, it will be readily appreciated that the details of the gas cabinet 202 as shown in FIG. 3 may readily apply to each one of the one or more gas cabinets 202 as shown in FIG. 2, respectively.

As shown in FIG. 3, the gas cabinet 202 includes a housing 216 and a door 218 in mechanical cooperation with the housing 216. For example, the door 218 may be hingedly coupled to the housing 216 such that the door 218 includes an opened position and a closed position. When the door 218 is in the opened position, a fluid container storage chamber 220 is readily accessible to an employee of a FAB such that the first fluid container 214a and the second fluid container 214b within the storage chamber 220 may be replaced by the employee when the first or second fluid container 214a, 214b, respectively, is empty. In some embodiments, the first and second fluid containers 214a, 214b contain the same type of specialty fluid. In some embodiments, the first and second fluid containers 214a, 214b contain different types of specialty fluids.

A first fluid container reception structure 222a, which may be referred to as a first gas container reception structure 222a, and a second fluid container reception structure 222b, which may be referred as a second gas container reception structure 222b, are present within the storage chamber 220. The first fluid container reception structure 222a receives the first fluid container 214a and the second fluid container reception structure 222b receives the second fluid container 214b. The one or more fluid pathways 208 may extend to corresponding ones of the first fluid container reception structures 222a and the second fluid container reception structures 222b within the gas cabinets 202 such that the fluid within the first and second fluid containers 214a, 214b within the gas cabinets 202 may be transported along the one or more fluid pathways 208 to the switch box 210.

A first fluid container valve 224a is at an upper end of the first fluid container 214a and a second fluid container valve 224b is at an upper end of the second fluid container 214b. The first and second fluid container valves 224a, 224b are configured to be opened to release the specialty fluid stored within the first and second fluid containers 214a, 214b, respectively. A first valve reception component 226 is in fluidic communication with the first fluid container 214a through the first fluid container valve 224a, and a second valve reception component 228 is in fluidic communication with the second fluid container valve 224b.

The specialty fluid stored within the first fluid container 214a may be mounted within the first fluid container 214a within the storage chamber 220 of the gas cabinet 202 as shown in FIG. 3 such that the first valve reception component 226 is in fluidic communication with the first fluid container valve 224a before the first fluid container valve 224a is opened to reduce the likelihood of exposure of contaminants to the specialty fluid either present within or previously present within the first fluid container 214a. The specialty fluid stored within the second fluid container 214b may be mounted within the second fluid container 214b within the storage chamber 220 of the gas cabinet 202 as shown in FIG. 3 such that the second valve reception component 228 is in fluidic communication with the second fluid container valve 224b before the second fluid container valve 224b is opened to reduce the likelihood of exposure of contaminants to the specialty fluid either present within or previously present within the second fluid container 214b.

A first fluid pathway 230 extends from the first valve reception component 226 to the test fluid pathway 208 as shown in FIG. 3. The specialty fluid from the first fluid container 214a may readily travel or pass through the first fluid pathway 230 to the test fluid pathway 208. For example, when the first fluid container valve 224a is opened, the specialty fluid exits the first fluid container 214a and passes through the first valve reception component 226. The specialty fluid may then travel or pass along and through the first fluid pathway when a first valve 234 and a second valve 236 are both opened such that the specialty fluid enters the test fluid pathway 208. The first valve 234 is along the first fluid pathway 230, and the second valve 236 is along the first fluid pathway and is closer to the test fluid pathway 208 relative to the first valve 234. In some embodiments, only one of the first valve 234 and the second valve 236 may be present along the first fluid pathway 230 such that the other one of the first valve 234 and the second valve 236 is not present.

A second fluid pathway 232 extends from the second valve reception component 228 to the test fluid pathway 208 as shown in FIG. 3. The specialty fluid from the second fluid container 214b may readily travel or pass through the second fluid pathway 232 to the test fluid pathway. For example, when the second fluid container valve 224b is opened, the specialty fluid exits the second fluid container 214b and passes through the second valve reception component 228. The specialty fluid may then travel and pass along or through the second fluid pathway 232 when a third valve 238 and a fourth valve 240 are both opened such that the specialty fluid enters the test fluid pathway 208. The third valve 238 is along the second fluid pathway 232, and the fourth valve 240 is present along the second fluid pathway 232 and is closer to the test fluid pathway 208 relative to the third valve 238. In some embodiments, only one of the third valve 238 and the fourth valve 240 may be present along the second fluid pathway 232 such that the other one of the third valve 238 and the fourth valve 240 is not present.

While not shown, in some embodiments, the first fluid pathway 230 may extend to a first one of the test fluid pathways 208, the second fluid pathway 232 may extend to a second one of the test fluid pathways 208, and the first one and the second one of the test fluid pathways 208 may be separate and distinct from each other such that the specialty fluid from the first fluid container 214a travels along and through the first one of the test fluid pathways 208 and the specialty fluid from the second fluid container 214b travels along and through the second one of the test fluid pathways 208. For example, the specialty fluid from the first fluid container 214a passes through and along the first one of the test fluid pathways 208 to the analyzer 204, and the specialty fluid from the second fluid container 214b passes through and along the second one of the test fluid pathways 208 to the analyzer 204. In other words, the specialty fluid from the first fluid container 214a does not travel along a fluid pathway along which the specialty fluid from the second fluid container 214b travels along and vice versa.

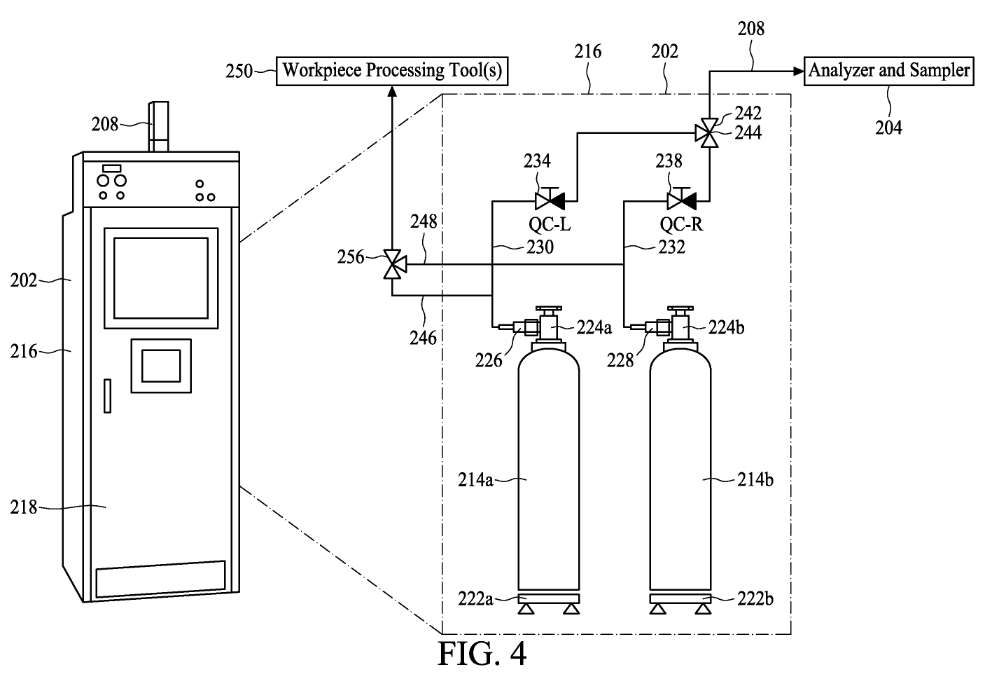

While not shown, in some embodiments, the first, second, third, and fourth valves 234, 236, 238, 240 may be replaced by a first three-way valve 242 at a location 244 (see FIG. 4 of the present disclosure) at which the first fluid pathway 230 and the second fluid pathway 232 meet up with the test fluid pathway 208. While not shown, in some embodiments, the second and fourth valves 236, 240 may be replaced by a three-way valve at the location 244 such that the three-way valve is present at the location 244 and the first and third valves 234, 238, respectively, are present along the first and second fluid pathways 230, 232, respectively, as well. In other words, positioning and a number of respective valves along the first and second fluid pathways 230, 232, respectively, may be varied in some embodiments of the gas cabinet 202 as shown in FIG. 3.

In view of the above discussion, it will be readily appreciated that the fluid pathways discussed herein may be formed utilizing one or more pipes to define the fluid pathways as discussed herein. In other words, the pipes may be fluidically coupled or in mechanical engagement with each other such that the pipes define the respective fluid pathways as discussed herein.

A third fluid pathway 246 extends from the first valve reception component 226 extends from the first valve reception component 224a, and a fourth fluid pathway 248 extends from the second valve reception component 228. The third and fourth fluid pathways 246, 248 extend to one or more workpiece processing tools 250. In some embodiments, the third and the fourth fluid pathways 246, 248 extend to a respective one of the one or more workpiece processing tools 250 such that the respective one of the one or more workpiece processing tools 250 may receive the specialty fluids present within either one of the first fluid container 214a and the second fluid container 214b within the gas cabinet 202. In some embodiments, the third fluid pathway 246 may extend to a first one of the one or more workpiece processing tools 250 such that the first one of the one or more workpiece processing tools 250 may receive the specialty fluid present within the first fluid container 214a, and the fourth fluid pathway 248 may extend to a second one of the one or more workpiece processing tools 250 separate and distinct from the first one of the one or more workpiece processing tools 250 such that the second one of the one or more workpiece processing tools 250 may receive the specialty fluid present within the second fluid container 214b.

A fifth valve 252 is present along the third fluid pathway 246. When the fifth valve 252 is opened, the specialty fluid from the first fluid container 214a may travel or pass along and through the third fluid pathway 246 and through the fifth valve 252 to the one or more workpiece processing tools 250.

A sixth valve 254 is present along the fourth fluid pathway 248. When the sixth valve 254 is opened, the specialty fluid from the second fluid container 214b may travel or pass along and through the fourth fluid pathway 248 and through the sixth valve 254 to the one or more workpiece processing tools 250.

While not shown in FIG. 3, in some embodiments, the fifth valve 252 and the sixth valve 254 may be replaced by a second three-way valve 256 (see FIG. 4 of the present disclosure). In other words, the third and fourth fluid pathways 246, 248 may both have ends in fluid communication with the second three-way valve 256 (see FIG. 4 of the present disclosure).

The one or more workpiece processing tools 250 may utilize the specialty fluid from the first and second fluid containers 214a, 214b to perform one or more types of processing or refinement techniques on one more workpieces. For example these processing or refinement techniques may include etching techniques, mask formation techniques, patterning techniques, or some similar or like type of workpiece processing or refinement techniques or combination of workpiece processing or refinement techniques that may be performed within a FAB.

FIG. 4 is directed to an example of an alternative embodiment of one of the one or more gas cabinets 202. The gas cabinet 202 as shown in FIG. 4 is the same or similar to the gas cabinet 202 as shown in FIG. 3; however, unlike the gas cabinet 202 as shown in FIG. 3, the gas cabinet 202 as shown in FIG. 4 includes the first three-way valve 242 and the second three-way valve 256. The first and second three-way valves 242, 256 in the gas cabinet 202 as shown in FIG. 4 are present instead of the second valve 236, the fourth valve 240, the fifth valve 252, and the sixth valve 254.

In alternative embodiments of the one or more gas cabinets 202, the one or more gas cabinets may have varying combinations of the first, second, third, fourth, fifth, and sixth valves 234, 236, 238, 240, 252, 254, the first three-way valve 242, and the second three-way valve 256. For example, in some alternative embodiments of the one or more gas cabinets 202, the gas cabinets 202 may only include the second three-way valve 256 along with the first, second, third, and fourth valves 234, 236, 238, 240. In other words, the respective valves 234, 236, 238, 240, 242, 256 may be present within various embodiments of the one or more gas cabinets in various combinations.

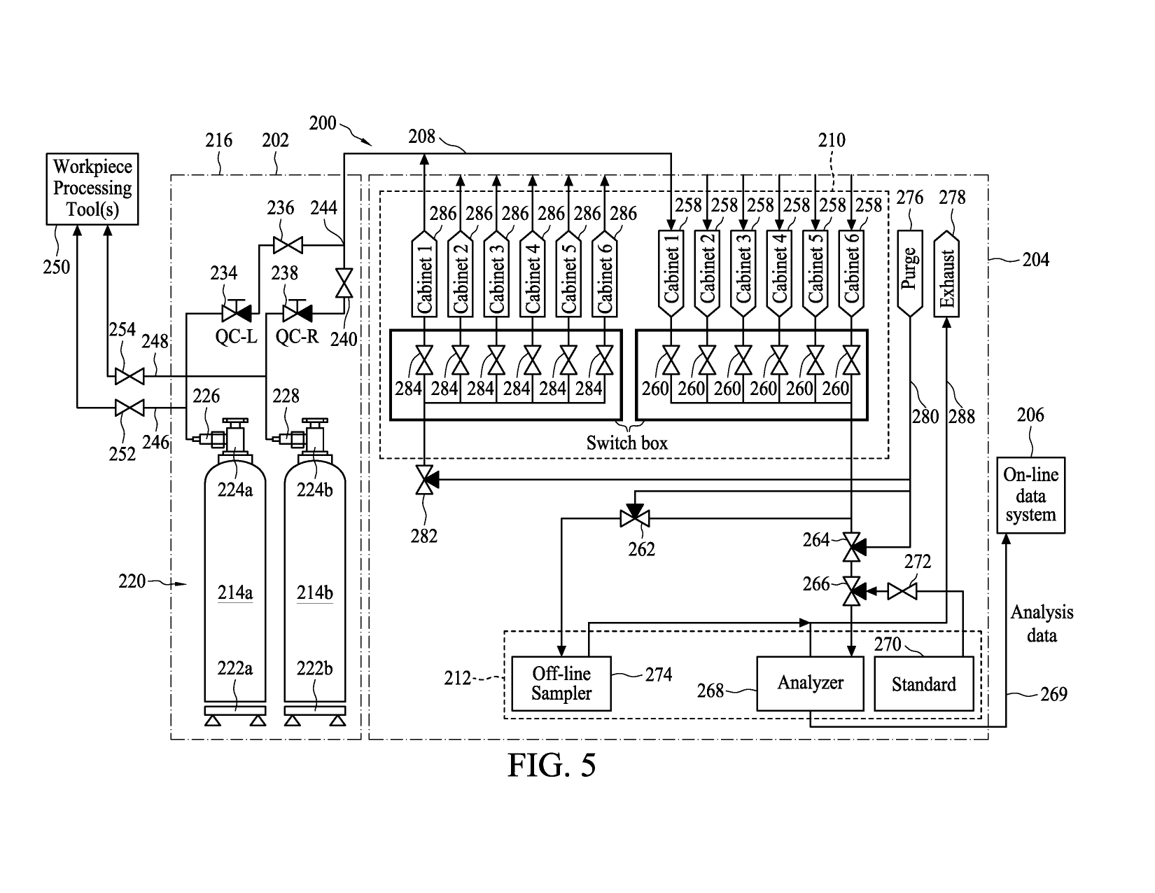

FIG. 5 illustrates an example of a detailed schematic view of the fluid analyzer and sampler 204, which may be a tool or a system, in fluidic communication with the embodiment of the example of the gas cabinet 202 as shown in FIG. 3 of the present disclosure. As shown in FIG. 5, the gas cabinet 202, which is the same as the gas cabinet 202 as shown in FIG. 3, is in fluid communication with a left-most switch box inlet 258 (e.g., labeled “Cabinet 1” as shown in FIG. 5) of the switch box 210 through the respective one of the one or more fluid pathways 208 as shown in FIG. 3. The fluid pathway 208 as shown in FIG. 3 is structured to allow specialty fluid from either one of or both of the first and second fluid containers 214a, 214b, respectively, to the switch box inlet 258 of the switch box 210. The switch box inlet 258 of the switch box is one of a plurality of switch box inlets 258 (e.g., labeled “Cabinet 1,” “Cabinet 2,” “Cabinet 3,” “Cabinet 4,” “Cabinet 5,” and “Cabinet 6”). Each one of the plurality of switch box inlets 258 as shown in FIG. 3 is in fluid communication with one of the one or more gas cabinets 202 through one of the one or more fluid pathways 208 as shown in FIG. 2.

Each one of the plurality of switch box inlets 258 as shown in FIG. 3 is in fluid communication with one of a plurality of switch box inlet valves 260, which are configured to control an amount of specialty fluid that is present within a corresponding one of the fluid pathways 208 and is to be introduced through the switch box into the testing tool 212 by opening or closing a corresponding one of the plurality of switch box inlet valves 260. For example, when the first and second valves 234, 236 are opened the specialty fluid travels from the first fluid container 214a through the first and second valves 234, 236 and into the fluid pathway 208 such that the specialty fluid then travels to the left-most switch box inlet 258, and the left-most switch box inlet valve 260 may then be opened such that the specialty fluid passes through the left-most switch box inlet valve 260 and is introduced into the testing tool 212.

A third three-way valve 262 and a fourth three-way valve 264 are in fluid communication with each one of the plurality of switch box inlet valves 260. The third and fourth three-way valves 262, 264 are downstream from the plurality of switch box inlet valves 260. A fifth three-way valve 266 is in fluid communication with the fourth three-way valve.

An analyzer 268 is downstream from the fifth three-way valve 266. For example, when one of the plurality of switch box inlet valves 260 is opened, the fourth and fifth three-way valves 264, 266 are opened, and the third three-way valve 262 is closed, specialty fluid may readily pass through the fourth and fifth three-way valves 264, 266 and be introduced to the analyzer 268 such that the analyzer 268 may perform one or more types of tests on the specialty fluid to determine characteristics (e.g., contaminant level, temperature, composition, etc.) of the specialty fluid introduced into the analyzer 268. The analyzer 268 may include one or more types of analysis tools, one or more types of sensors, or one or more other similar or like components for performing one or more types of tests to determine characteristics of specialty fluids introduced to the analyzer 268. These types of tests may include spectroscopy tests chromatography tests, spectrometry tests, or may be some other similar or like tests that may be performed by the analyzer 268 on specialty fluid received by the analyzer 268 from at least one of the one or more gas cabinets 202. For example, these types of tests may include cavity ring-down spectroscopy (CRDS), a gas chromatography test with a pulsed discharge ionization detector (PDHID) detector (GC-PDHID), a gas chromatography-mass spectrometry (GC-MS) test, an ion chromatography-mass spectrometry (IC-MS) test, a Fourier transform infrared (FTIR) test, a time-of-flight mas spectrometry (ToFMS) test, or some other similar or like tests that may be performed on a fluid such as a liquid or gas that is received by the analyzer 268.

The on-line data system 206 is in electrical communication with the analyzer 268 as shown in FIG. 5. The on-line data system 206 may receive data signals 269 from the analyzer 268 that is representative of characteristics (e.g., contaminant levels, compositional make up, temperature, moisture levels, etc.) of the specialty fluid that is tested by the analyzer 268. In some embodiments, the on-line data system 206 may include a memory, a processor, a display, a user interface, and similar or like electrical components that allow the on-line data system 206 to store data based on the data signals 269 received from the analyzer 268, process the data signals 269 received from the analyzer 268, or display data stored within the on-line data system 206 based on the data signals 269 received from the analyzer 268. For example, the data stored on a memory (not shown) of the on-line data system 206 may be readily accessible and visible to an employee of the FAB through a display (not shown) utilizing a user interface in electrical communication with the memory of the on-line data system 206. The data stored in the memory of the on-line data system 206 may include data representative of contaminant levels of the specialty fluid, temperature of the specialty fluid, composition of the specialty fluid, moisture levels within the specialty fluid, or some other similar or like type of data with respect to physical characteristics of the specialty fluid tested by the analyzer 268.

This data stored within the memory of the on-line data system 206 may be utilized such that the employee may monitor the physical characteristics of the specialty fluid in real time. For example, a processor (not shown) of the on-line data system 206 may be utilized to process the data signals 269 to convert the data signals 269 into data or processed data to be stored on the memory of the on-line data system. The processor of the on-line data system 206 may utilize the data signals 269 or the data to determine whether a notification should be output on the display. The notification may be a warning notification that indicates that a characteristic of the specialty fluid is outside a selected tolerance or tolerances to avoid introducing the specialty fluid with the characteristic outside the selected tolerance or tolerances to the workpiece processing tools 250. For example, the employee through the user interface of the on-line processing tool 206 may seize introduction of the specialty fluid with the characteristic outside the selected tolerances to the workpiece processing tools 250 to avoid introducing the specialty fluid of insufficient quality to workpieces within the workpiece processing tools 250. Alternatively, the processor of the on-line data system 206 may automatically prevent the introduction of the specialty fluid of insufficient quality to the workpieces within the workpiece processing tools 250. Reducing the likelihood of the introduction of the specialty fluid of insufficient quality when the characteristic of the specialty fluid is outside the selected tolerance or tolerances, reduces a number of workpieces that become scrap or waste and increases a yield number of manufactured components (e.g., semiconductor dice, semiconductor packages, semiconductor components, etc.) utilizing the FAB.

In some embodiments, the warning notification may be output when a contaminant level of the specialty fluid tested by the analyzer 268 is greater than or equal to a contaminant level threshold. In some embodiments, the warning notification may be output when the contaminant level of the specialty fluid tested by the analyzer 268 is only greater than the contaminant level threshold. While the above discussion is with respect to outputting the warning notification to avoid introducing the specialty fluid of insufficient quality to workpieces within the workpiece processing tools 250 with respect to the contaminant level of the specialty fluid, it will be readily appreciated that the characteristic monitored by the on-line data system may include other characteristics of the specialty fluid or combination of characteristics of the specialty fluid. For example, as discussed earlier herein, these characteristics may include contaminant levels, compositional make up, temperature, moisture levels.

The data collected by the on-line data system 206 by testing the specialty fluids received by the analyzer 268 may be utilized to compile real-world data in real-world data sheets. The real-world data compiled in these real-world data sheets may be compared to data in the manufacturer technical specifications to determine whether the specialty fluids tested by the analyzer 268 have characteristics similar to those as indicated and asserted by the data with respect to quality of the specialty fluid within the manufacturer technical specifications. This real-world data may be utilized to determine the quality of the specialty fluids from various manufacturers to determine which manufacturers provide specialty fluids of consistent quality to reduce the likelihood of introducing specialty fluids of insufficient quality to workpieces within the workpiece processing tools 250. In other words, the collection of this real-world data can be utilized to verify whether the data with respect to the quality of the specialty fluids provided in the manufacturer technical specifications is accurate, and, therefore, this allows a customer to limit purchasing specialty fluids only from manufacturers that provide specialty fluids of sufficient quality and sufficient consistency to reduce the likelihood of the introduction of specialty fluids of insufficient quality to workpieces within the workpiece processing tools 250.

While not shown, the on-line data system 206 may be in electrical communication with one or more processors within the FAB. For example, the on-line data system 206 may be in electrical communication either in a wired or a wireless fashion such that an employee in a control room of the FAB may be able to access the data stored within the on-line data system 206. Similar to how the processor of the on-line data system 206 outputs a notification or warning when a characteristic of specialty fluid that is tested by the analyzer 268 is outside a selected tolerance or tolerances, the processor of the on-line data system 206 may output a notification or warning to a display (not shown) within the control room such that the employee within the control room may take actions and steps to prevent the introduction of the specialty fluid of insufficient quality into the workpiece processing tools 250. Alternatively, the processor of the on-line data system 206 may output the notification or warning to the display (not shown) within the control room and the processor of the on-line data system 206 may automatically seize utilization of the specialty fluid of insufficient quality to avoid introduction of the specialty fluid of insufficient quality into the workpiece processing tools 250. Again, reducing the likelihood of the introduction of the specialty fluid of insufficient quality when the characteristic of the specialty fluid is outside the selected tolerance or tolerances, reduces a number of workpieces that become scrap or waste and increases a yield number of manufactured components (e.g., semiconductor dice, semiconductor packages, semiconductor components, etc.) utilizing the FAB.

The testing tool 212 further includes a standard calibration device 270 as shown in FIG. 5. The standard calibration device 270 contains a standardized fluid that may be utilized to calibrate the one or more testing tools (not shown), the one or more types of sensors, and the one or more testing components within the analyzer 268. For example, the standard calibration device 270 may contain the standardized fluid of which the compositional and physical characteristics are already known such that the standardized fluid may be utilized to perform a calibration process on the analyzer 268 to calibrate the analyzer 268. The standardized fluid may be introduced into the analyzer 268 to perform the calibration process by opening a standardized fluid valve 272 and opening the fifth three-way valve 266 such that the standardized fluid travels along a standardized fluid pathway that extends from the standard calibration device 270 through the standardized fluid valve 272 and the fifth three-way valve 266 to the analyzer 268.

The testing tool 212 further includes an off-line sampler 274 as shown in FIG. 5. The off-line sampler 274 may be utilized to collect a sample of a specialty fluid that may need to be transported away from the testing tool 212 to perform another type of test that the analyzer 268 may not be configured to conduct. For example, the off-line sampler 274 may collect the sample of the specialty fluid by opening the third three-way valve 262 and closing the fourth three-way valve 264 such that specialty fluid may travel along a fluid pathway that extends from one of the one or more switch box inlets 258 through the third three-way valve 262 to the off-line sampler 274. The sample may be collected in a removable storage container (not shown) within the off-line sampler 274 such that the sample of the specialty fluid is stored within the removable storage container. A robot or an employee may then remove the removable storage container from the off-line sampler 274 and transport the removable storage container, which contains the sample of the specialty fluid, to an external testing tool or testing area external to the testing tool 212. One or more tests may then be performed on the sample stored within the removable storage container. These one or more tests may not be capable of being carried out by the one or more types of testing tools, the one or more types of sensors, and the one or more testing components within the analyzer 268.

The fluid analyzer and sampler 204 further includes a purge fluid inlet 276 and an exhaust fluid outlet 278. The purge fluid inlet 276 is in fluid communication with a purge fluid source (not shown) such that a purge fluid may pass through the purge fluid inlet 276 when opened and travel along a purge fluid pathway 280. The purge fluid inlet 276 is in fluid communication with the third three-way valve 262, the fourth three-way valve 264, and a sixth three-way valve 282 through the purge fluid pathway 280 such that the purge fluid may be provided to the analyzer 268, the off-line sampler 274, and a plurality of switch box outlet valves 284. Each one of the plurality of switch box outlet valves 284 is in fluid communication with a corresponding one of a plurality of switch box outlets 286. Each one of the plurality of switch box outlets 286 is in fluid communication with a corresponding one of the test fluid pathways 208 that are each in fluid communication with a corresponding one of the one or more gas cabinets 202. The purge fluid may be introduced into the analyzer by opening the purge fluid inlet 276, opening the fourth three-way valve 264, and opening the fifth three-way valve 266. The purge fluid may be introduced into the off-line sampler by opening the purge fluid inlet 276 and opening the third three-way valve 262. The purge fluid may be introduced to the plurality of switch box outlet valves 284 by opening the purge fluid inlet 276 and opening the sixth three-way valve, and the purge fluid may then be introduced into at least one of the fluid pathways 208 by opening at least one of the plurality of switch box outlet valves 284. The purge fluid may be introduced into the fluid pathways 208 to sterilize one or more of the fluid pathways 208 before or after specialty fluids are transported through the one or more fluid pathways to the analyzer 268 or the off-line sampler 274 such that these specialty fluids are not contaminated along the fluid pathways 208 before reaching the analyzer 268 or the off-line sampler 274, respectively. The purge fluid may be an ultra-pure clean water (UPC), may be a nitrogen based fluid, or may be some other suitable type of purge fluid.

The exhaust fluid outlet 278 is in fluid communication with the analyzer 268 and the off-line sampler 274, respectively, along an exhaust fluid pathway 288. For example, after a specialty fluid is introduced into the analyzer 268 and the analyzer 268 has performed one or more types of tests on the specialty fluid, the specialty fluid may then exit the analyzer through the exhaust fluid pathway 288 and travel from the analyzer 268 to the exhaust fluid outlet 278. The specialty fluid that was previously tested by the analyzer 268 may exit the analyzer and sampler 204 through the exhaust fluid outlet 278 such that the specialty fluid that was previously tested may be disposed. Alternatively, the purge fluid introduced to purge the off-line sampler 274 and the analyzer 268 may exit the off-line sampler 274 and the analyzer 268 through the exhaust fluid pathway 288, and exit the analyzer and sampler 204 through the exhaust fluid outlet 278. While not shown, in some embodiments, the exhaust fluid outlet 278 may be in fluid communication with the plurality of switch box inlet valves 260 and the plurality of switch box outlet valves 284, respectively, such that excess fluid or left over fluid may readily exit through the exhaust fluid outlet 278. In other words, the exhaust fluid outlet 278 may be in fluid communication with various fluid pathways and components of the system 200 such that fluid may readily exit the system 200 through the exhaust fluid outlet 278.

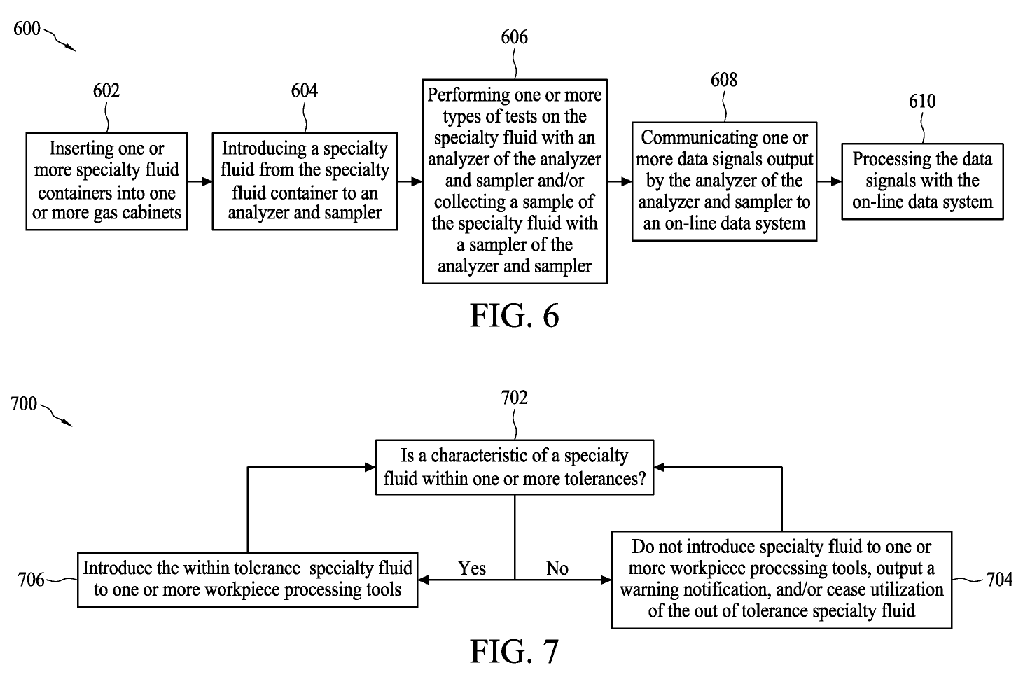

FIG. 6 is directed to a flowchart 600 of a method of utilizing the analyzer and sampler 204 (see FIG. 5) of the system 200 (see FIG. 2) to test specialty fluids within the one or more specialty fluid containers 214a, 214b (see FIG. 5) stored in the one or more gas cabinets 202 (see FIG. 2). The method illustrated in the flowchart 600 as shown in FIG. 6 is advantageous over the method illustrated in the flowchart 122 as shown in FIG. 1B, which will be discussed in further detail later herein.

In a first step 602 of the method as illustrated in the flowchart 600, one or more respective ones of the specialty fluid containers 214a, 214b are inserted into respective ones of the one or more gas cabinets 202. For example, two of the specialty fluid containers 214a, 214b may be inserted into each one of the one or more gas cabinets 202 such that each of the six gas cabinets 202 as in the embodiment of the system 200 as shown in FIG. 2 each contains two of the specialty fluid containers 214a, 214b, respectively. In other words, similar to as shown in FIG. 5, in which the gas cabinet 202 as shown in FIG. 5 contains a pair of the specialty fluid containers 214a, 214b, each of the other ones of the one or more gas cabinets 202 also contains a pair of the specialty fluid containers 214a, 214b. These specialty fluid containers 214a, 214b may be installed into the gas cabinets 202 by an employee of the FAB.

After the first step 602 in which the one or more specialty fluid containers 214a, 214b are inserted and installed into the one or more gas cabinets 202, in a second step 604 of the flowchart 600 a specialty fluid from one of the specialty fluid containers 214a, 214b is introduced to the analyzer and sampler 204 through the switch box 210. For example, a specialty fluid from one of the specialty fluid containers 214a, 214b in the left-most one of the gas cabinets 202 as shown in FIG. 2 is introduced into the analyzer 268 by opening the left-most one of the plurality of switch box inlet valves 260, opening the fourth three-way valve 264, and opening the fifth three-way valve 266 such that the specialty fluid passes through the switch box 210, the fourth three-way valve 264, and the fifth three-way valve 266 along the respective fluid pathway that extends to the analyzer 268. Alternatively, the specialty fluid from one of the specialty fluid containers 214a, 214b in the left-most one of the gas cabinets as shown in FIG. 2 is introduced into the off-line sampler 274 by opening the third three-way valve 262 along with opening the left-most one of the plurality of switch box inlet valves 260 such that the specialty fluid travels along the respective fluid pathway and through the third three-way valve 262 to the off-line sampler 274.

After the second step 604 in which the specialty fluid is introduced to the analyzer and sampler 204 and to either one of the analyzer 268 and the off-line sampler 274 or both, in a third step 606 the specialty fluid is tested by the analyzer 268, a sample of the specialty fluid is collected by the off-line sampler 274, or the specialty fluid is both tested by the analyzer 268 and the sample of the specialty fluid is collected by the off-line sampler 274. For example, when the specialty fluid is communicated and provided to both the analyzer 268 and the off-line sampler 274, the analyzer 268 performs one or more tests on the specialty fluid and the off-line sampler 274 collects a sample of the specialty fluid. Alternatively, if the specialty fluid is only introduced to the analyzer 268, the analyzer 268 performs one or more tests on the specialty fluid and the off-line sampler 274 does not collect the sample of the specialty fluid. Alternatively, if the specialty fluid is only introduced to the off-line sampler 274, the off-line sampler 274 collects the sample of the specialty fluid and the analyzer 268 does not perform one or more tests on the specialty fluid. For the purposes of the following steps in the flowchart, the analyzer 268 receives the specialty fluid.

After the third step 606 in which either one of or both the analyzer 268 and the off-line sampler 274 receives the specialty fluid, in a fourth step 608 the analyzer 268 performs one or more tests on the specialty fluid when the analyzer 268 receives the specialty fluid. These one or more tests may include cavity ring-down spectroscopy (CRDS), a gas chromatography test with a pulsed discharge ionization detector (PDHID) detector (GC-PDHID), a gas chromatography-mass spectrometry (GC-MS) test, an ion chromatography-mass spectrometry (IC-MS) test, a Fourier transform infrared (FTIR) test, a time-of-flight mas spectrometry (ToFMS) test, or some other similar or like tests that may be performed on the specialty fluid, which may be in a liquid-state, semi-liquid state, semi-gaseous state, or gaseous state that is received by the analyzer 268. Upon completion of these one or more tests, the analyzer 268 outputs one or more data signals 269 to the on-line data system 206 based on results of the one or more tests performed on the specialty fluid.

After the fourth step 608 in which the analyzer 268 performs one or more tests on the specialty fluid and outputs the data signals 269 to the on-line data system 206, in a sixth step the on-line data system 206 processes the one or more data signals 269 output and received from the analyzer 268. For example, as discussed earlier, the on-line data system 206 may include a processor (e.g., CPU, microprocessor, or some other similar or suitable type of processor) that processes the one or more data signals 269 and stores data collected based on the processing of the data signals 269 on a memory of the on-line data system 206. The processor of the on-line data system 206 may perform real time analysis utilizing the one or more data signals 269 to determine if the specialty fluid is within selected tolerances. This real time analysis performed by the on-line data system 206 will be discussed in further detail with respect to a flowchart 700 illustrating a control block diagram as shown in FIG. 7. The data stored on the memory of the on-line data system 206 may be utilized to generate one or more real time specialty fluid specifications, which may be compared to a manufacturer technical specification to verify or determine if the manufacturer technical specification with respect to the specialty fluid tested is accurate and consistent. By continuously collecting, monitoring, and comparing the real time data collected by the on-line data system 206 through testing specialty fluids with the analyzer 268, the quality and consistency of specialty fluids provided by various manufacturers of the specialty fluids may be monitored in real time to determine which of the manufacturers of the specialty fluids consistently provide specialty fluids of sufficient quality for utilization within the FAB. Monitoring which manufacturers of the specialty fluids provide sufficient quality specialty fluids the most consistently allows the FAB to only purchase from those manufacturers that consistently (e.g., almost always) provide specialty fluids of sufficient quality to reduce the generation of waste and scrap by exposing workpieces to specialty fluid that is of insufficient quality.

As should be readily appreciated, the on-line data system 206 is “on-line” as the on-line data system 206 collects and processes real time data signals 269 from the analyzer 268 to determine real time data with respect to the specialty fluids tested by the analyzer 268. In other words, unlike the off-line data system 120, the on-line data system 206 is in electrical communication with the analyzer 268 of the system 200, which is fluidically on-line and hooked up to the one or more workpiece processing tools 250 of the FAB such that the system 200 is “on-line” and the on-line data system 206 is “on-line.”

FIG. 7 illustrates the flowchart 700 of the control block diagram to monitor and test specialty fluids within the specialty fluid containers 214a, 214b within the one or more gas cabinets 202 in real time and determine whether the specialty fluids are of sufficient quality to be introduced to the one or more workpiece processing tools 250 to reduce the likelihood of generating waste or scrap by exposing the workpieces within the workpiece processing tools to specialty fluid of insufficient quality. For example, this process as shown in the flowchart 700 may be carried out by the processor of the on-line data system 206 in the fifth step 610 of the method as shown in the flowchart 600.

In at least one embodiment of the control block diagram of the flowchart 700, after the data signals 269 output by the analyzer 268 are received by the processor of the on-line data system 206, in a first block 702 of the flowchart 700 the processor of the on-line data system 206 may process those data signals 269 such that data is collected and analyzed by the processor of the on-line data system 206. One such type of analysis may be determining whether a specialty fluid tested by the analyzer 268 is within one or more tolerances or satisfies one or more pre-determined value. For example, these one or more tolerances or the one or more pre-determined value may include at least one of a contaminant level threshold, a temperature threshold, a compositional make up, or some other type of tolerance, pre-determined value, or characteristic of the specialty fluid that is analyzed by the analyzer 268 to determine if the specialty fluid is of sufficient quality and within the one or more tolerances. For purposes of brevity and simplicity of the present disclosure, the following discussion will focus on when the one or more tolerances or the one or more pre-determined values includes a contaminant level threshold.

When the one or more tolerances or one or more pre-determined values includes or is the contaminant level threshold, in the fifth step 610 of the flowchart 600, the analyzer 268 performs one or more tests on the specialty fluid received from at least one of the one or more gas cabinets 202, the processor of the on-line data system 206 processes the data signals 269 output by the analyzer 268, and the processor of the on-line data system 206 compares a contaminant level measured by the analyzer 268 to the contaminant threshold. In some embodiments, if the contaminant level measured by the analyzer 268 is greater than the contaminant level threshold, then the flowchart 700 proceeds to a second block 704, and, alternatively, if the contaminant level measured by the analyzer 268 is less than or equal to the contaminant level threshold, then the flow chart 700 proceeds to a third block 706 instead. In some alternative embodiments, if the contaminant level measured by the analyzer 268 is greater than or equal to the contaminant level threshold, then the flowchart 700 proceeds to the second block 704, and, alternatively, if the contaminant level measured by the analyzer 268 is less than the contaminant level threshold, then the flowchart 700 proceeds to the third block 706 instead. While not discussed in detail, it will be readily appreciated that this comparison between the contaminant level measured by the analyzer and the contaminant level threshold may readily apply to any other number of types of tolerances (e.g., temperature, compositional make up, etc.).

When the contaminant level measured by the analyzer 268 is outside the selected contaminant level threshold, the flowchart 700 proceeds to the second block 704. In the second block 704, the processor of the on-line data system 206 may be configured to prevent or seize utilization of the specialty fluid of insufficient quality to avoid introduction of the specialty fluid of insufficient quality to one or more workpieces within the one or more workpiece processing tool 250. For example, the on-line data system 206 may be in electrical communication with a controller or one or more actuators that control the fifth valve 252 and the sixth valve 254. For example, the on-line data system 206 provides an instruction signal to the controller or the one or more actuators such that either one of or both the fifth valve 252 and the sixth valve 254 are closed, respectively, or remained closed, respectively, to avoid and prevent the introduction of the specialty fluid of insufficient quality to the one or more workpiece processing tools 250. Alternatively, when the second three-way valve 256 is present instead of the fifth and sixth valves 252, 254, the on-line data system 206 may be in electrical communication with a controller or an actuator that controls the second three-way valve 256 such that the on-line data system 206 provides an instruction signal to the controller or the actuator. For example, the on-line data system 206 provides an instruction signal to the controller or the actuator such that the second three-way valve 256 is closed or remains closed, respectively, to avoid and prevent the introduction of the specialty fluid of insufficient quality to the one or more workpiece processing tools 250.

When the contaminant level measured by the analyzer 268 is outside the selected contaminant level threshold, in the second block 704, the processor of the on-line data system 206 may output a warning notification to a display of the on-line data system 206 or a display within a control room of the FAB readily visible by an employee of the FAB such that the employee is notified that the specialty fluid is of insufficient quality within a respective one of the one or more specialty fluid containers 214a, 214b within one of the one or more gas cabinets 202. This warning notification visible to the employee of the FAB may include an indication of which specific one of the one or more specialty fluid containers 214a, 214b may contain the specialty fluid of insufficient quality such that the employee of the FAB may replace the specialty fluid containers 214a, 214b with a new one of the specialty fluid containers 214a, 214b that should contain specialty fluid of sufficient quality. In some embodiments, once the specialty fluid containers 214a, 214b containing the specialty fluid of insufficient quality are replaced with new specialty fluid containers 214a, 214b that should contain specialty fluid of sufficient quality, the processor of the on-line data system 206 may automatically detect that the replacement has occurred and proceed back to the first block 702 of the flowchart 700. In some alternative embodiments, the employee of the FAB may need to provide an input through a user interface of the on-line data system 206 or in the control room of the FAB such that the flowchart proceeds from the second block 704 back to the first block 702.

When the contaminant level measured by the analyzer 268 of the specialty fluid is less than the contaminant level threshold or, in some embodiments, equal to the contaminant level threshold, the processor of the on-line data system 206 determines that the specialty fluid is of sufficient quality such that the specialty fluid is introduced to the one or more workpiece processing tools 250 for processing or refining one or more workpieces. For example, the on-line data system 206 may be in electrical communication with a controller or one or more actuators that control the fifth valve 252 and the sixth valve 254. For example, the on-line data system 206 provides an instruction signal to the controller or the one or more actuators such that either one of or both the fifth valve 252 and the sixth valve 254 are opened, respectively, or remain opened, respectively, to introduce of the specialty fluid of sufficient quality to the one or more workpiece processing tools 250 for processing or refining of the one or more workpieces with the one or more workpiece processing tools 250. Alternatively, when the second three-way valve 256 is present instead of the fifth and sixth valves 252, 254, the on-line data system 206 may be in electrical communication with a controller or an actuator that controls the second three-way valve 256 such that the on-line data system 206 provides an instruction signal to the controller or the actuator. For example, the on-line data system 206 provides an instruction signal to the controller or the actuator such that the second three-way valve 256 is opened or remains opened, respectively, to introduce the specialty fluid of sufficient quality to the one or more workpiece processing tools 250 for processing of one or more workpieces with the one or more workpiece processing tools 250.

After the introduction of the specialty fluid of sufficient quality to the one or more workpiece processing tools 250, the flowchart 700 reverts from the third block 706 to the first block 702 accordingly. The above process of the control block diagram as shown in the flowchart 700 of FIG. 7 is performed in succession over and over again to monitor the quality of the specialty fluid to be introduced to the one or more workpiece processing tools 250 such that the specialty fluid quality is determined in real time. This real time monitoring of the quality of the specialty fluid reduces the likelihood of exposing one or more workpieces being processed by the one or more workpiece processing tools 250 to specialty fluid of insufficient quality that may degrade the quality of the one or more workpieces.

In view of the above discussion, the control block diagram as shown in the flowchart 700 of FIG. 7 allows for the on-line data system 206 or some other processor external to the analyzer and sampler 204 to determine whether the specialty fluid is within a selected tolerances such that the specialty fluid is of sufficient quality before being supplied to the one or more workpiece processing tools 250. This reduces the likelihood of or completely avoids exposing one or more workpieces within the one or more workpiece processing tools 250 to the specialty fluid when the specialty fluid is of insufficient quality, which, in turn, may reduce waste and scrap costs absorbed by the FAB. In some alternative embodiments, the analyzer and sampler 204 may include a processor that is in electrical communication with the analyzer 268 and is configured to process the data signals 269 output by the analyzer 268.

In view of the discussions within the present disclosure, it will be readily appreciated that monitoring the real time quality of the specialty fluids introduced to one or more workpiece processing tools 250 decreases waste and scrap costs and allows for monitoring of consistency of the quality of the specialty fluids provided by manufacturers of the specialty fluid. Waste and scrap costs absorbed by the FAB are reduced as specialty fluids of insufficient quality are not introduced to the one or more workpiece processing tools 250 such that the workpieces are not degraded by being exposed to the specialty fluids of insufficient quality. This decreases the number of semiconductor products that are of insufficient quality to be provided to customers, and, in turn, increases a yield of a number of semiconductor products of sufficient quality to be provided by customers. In other words, the units per hour (UPH) of the FAB is increased by avoiding or preventing the introduction of specialty fluids of insufficient quality to workpieces being processed by the one or more workpiece processing tools 250. Consistency of specialty fluids provided by manufacturers may be monitored as the data collected by the on-line data system 206 by performing one or more tests on specialty fluids in real time with the analyzer 268 may be compared to manufacturer technical specifications with respect to the specialty fluids to determine whether the manufacturers are providing specialty fluids of sufficient quality for utilization within the FAB and to be introduced to the one or more workpiece processing tools. Monitoring this consistency of quality of specialty fluid provided by various manufacturers of the specialty fluids allows the FAB to reduce costs as the specialty fluids may only be repurchased from manufacturers that provide specialty fluids of sufficient quality almost all of the time reducing the likelihood of introducing specialty fluid of insufficient quality into the one or more workpiece processing tools 250.

A system may be summarized as including an on-line analysis system; a workpiece processing tool; a gas cabinet including a gas container reception structure; an analysis fluid pathway extends from the gas container reception structure to the on-line analysis system, wherein the gas container reception structure is in fluid communication with the on-line analysis system through the analysis fluid pathway; and a processing fluid pathway extends from the gas container reception structure to the workpiece processing tool, wherein the gas container reception structure is in fluid communication with the workpiece processing tool through the processing fluid pathway.

A method may be summarized as including moving a fluid from within a gas container within a gas cabinet along a fluid analysis pathway to an end of the fluid analysis pathway at a switch box of an on-line analysis system; moving the fluid through the switch box to an on-line analysis tool of the on-line analysis system by opening the switch box at the end of the fluid analysis pathway allowing the fluid to pass through the switch box to the on-line analysis tool; performing one or more types of tests on the fluid with one or more sensors of the on-line analysis tool of the on-line analysis system; generating one or more data signals based on results of the one or more types of tests on the fluid; receiving the one or more data signals at an on-line data system in electrical communication with the on-line analysis system; and processing a workpiece when the data signals satisfy a pre-determined value.

A specialty on-line analysis system may be summarized as including a purge fluid source containing a purge fluid; a switch box including: a plurality of fluid pathways; a plurality of first valves, each one of the plurality of first valves in fluid communication with one of the plurality of fluid pathways, each one of the plurality of first valves having a closed position and an opened position, and, during operation, one of the plurality of first valves is opened to allow a fluid from one of the plurality of fluid pathways to pass through the switch box; and a plurality of second valves, each one of the plurality of second valves in fluid communication with one of the plurality of fluid pathways and in fluid communication with the purge fluid source, each one of the plurality of second valves having a closed position and an opened position, and, during operation, one of the plurality of second valves is opened to allow the purge fluid to pass through the switch box and into one of the plurality of fluid pathways; an on-line analysis tool in fluid communication with the plurality of first valves; an off-line sampler tool in fluid communication with the plurality of first valves; a calibration fluid source containing a calibration fluid in fluid communication with the on-line analysis tool; and an exhaust port in fluid communication with the on-line analysis tool.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method, comprising:
moving a fluid from within a gas container within a gas cabinet along a fluid analysis pathway to an end of the fluid analysis pathway at a switch box of an on-line analysis system;
moving the fluid through the switch box to an on-line analysis tool of the on-line analysis system by opening the switch box at the end of the fluid analysis pathway allowing the fluid to pass through the switch box to the on-line analysis tool;
performing one or more types of tests on the fluid with one or more sensors of the on-line analysis tool of the on-line analysis system;
generating one or more data signals based on results of the one or more types of tests on the fluid;
receiving the one or more data signals at an on-line data system in electrical communication with the on-line analysis system; and
processing a workpiece when the data signals satisfy a pre-determined value.

2. The method of claim 1, further comprising outputting a notification when the fluid has a contamination level greater than a selected contamination level threshold.

3. The method of claim 2, further comprising replacing the gas container with a new gas container in response to the notification.

4. The method of claim 2, further comprising purging the fluid analysis pathway by passing a purge fluid through the fluid analysis pathway.

5. A specialty on-line analysis system, comprising:
a purge fluid source containing a purge fluid;
a switch box including:
a plurality of fluid pathways;
a plurality of first valves, each one of the plurality of first valves in fluid communication with one of the plurality of fluid pathways, each one of the plurality of first valves having a closed position and an opened position, and, during operation, one of the plurality of first valves is opened to allow a fluid from one of the plurality of fluid pathways to pass through the switch box; and
a plurality of second valves, each one of the plurality of second valves in fluid communication with one of the plurality of fluid pathways and in fluid communication with the purge fluid source, each one of the plurality of second valves having a closed position and an opened position, and, during operation, one of the plurality of second valves is opened to allow the purge fluid to pass through the switch box and into one of the plurality of fluid pathways;
an on-line analysis tool in fluid communication with the plurality of first valves;
an off-line sampler tool in fluid communication with the plurality of first valves;
a calibration fluid source containing a calibration fluid in fluid communication with the on-line analysis tool; and
an exhaust port in fluid communication with the on-line analysis tool.

6. The system of claim 5, wherein the purge fluid source is in fluid communication with the off-line sampler tool and the on-line analysis tool.

7. The system of claim 5, wherein, during operation, the calibration fluid is communicated from the calibration fluid source to the on-line analysis tool.

8. The system of claim 6, wherein the on-line analysis tool includes one or more sensors configured to perform a plurality of on-line tests.

9. A method of controlling delivery of a specialty fluid in a semiconductor fabrication facility, the method comprising:
receiving, at an on-line data system, one or more data signals from an on-line analysis tool, the one or more data signals representing one or more measured characteristics of a specialty fluid from a gas container installed in a gas cabinet;
comparing, by a processor of the on-line data system, the one or more measured characteristics to one or more thresholds;
determining, based on the comparing, whether the specialty fluid satisfies the one or more thresholds; and
in response to determining that the specialty fluid satisfies the one or more thresholds, providing an instruction signal from the on-line data system to open a valve along a processing fluid pathway coupled between the gas cabinet and a workpiece processing tool, thereby permitting the specialty fluid to be delivered to the workpiece processing tool.

10. The method of claim 9, further comprising, in response to determining that the specialty fluid does not satisfy the one or more thresholds, providing an instruction signal from the on-line data system to close the valve along the processing fluid pathway or to maintain the valve in a closed position, thereby preventing the specialty fluid from being delivered to the workpiece processing tool.

11. The method of claim 10, further comprising outputting a warning notification to a display, the warning notification identifying the gas cabinet and the gas container containing the specialty fluid that does not satisfy the one or more thresholds.

12. The method of claim 11, further comprising replacing the gas container with a replacement gas container in response to the warning notification.

13. The method of claim 12, further comprising, after replacing the gas container, routing a replacement specialty fluid from the replacement gas container to the on-line analysis tool and repeating the comparing.

14. The method of claim 9, wherein the one or more thresholds include at least one of a contaminant level threshold, a temperature threshold, a moisture level threshold, or a composition threshold.

15. The method of claim 9, further comprising storing the one or more measured characteristics in a memory of the on-line data system.

16. The method of claim 15, further comprising generating one or more real-time specialty fluid specifications based on the one or more measured characteristics stored in the memory.

17. The method of claim 16, further comprising comparing the one or more real-time specialty fluid specifications to a manufacturer technical specification associated with the specialty fluid.

18. The method of claim 9, further comprising, prior to receiving the one or more data signals, routing the specialty fluid from the gas container through an analysis fluid pathway to a switch box, and selectively routing the specialty fluid from the switch box to the on-line analysis tool by opening a switch box inlet valve corresponding to the gas cabinet.

19. The method of claim 18, further comprising purging the analysis fluid pathway by routing a purge fluid from a purge fluid source through the switch box and into the analysis fluid pathway.

20. The method of claim 9, further comprising calibrating the on-line analysis tool by routing a calibration fluid from a calibration fluid source to the on-line analysis tool.
